(19) United States
(12) Patent Application Publication
(10) Pub. No.: US 2025/0275824 A1
(43) Pub. Date: Sep. 4, 2025

(54) APPARATUS AND METHODS FOR TARGETED NAVIGATION

(71) Applicants: CANON U.S.A., INC., MELVILLE, NY (US); THE BRIGHAM AND WOMEN'S HOSPITAL INCORPORATED, BOSTON, MA (US)

(72) Inventors: Fumitaro Masaki, Brookline, MA (US); Takahisa Kato, Brookline, MA (US); Nobuhiko Hata, Waban, MA (US); Satoshi Kobayashi, Fukuoka (JP); Franklin King, Boston, MA (US); Brian Ninni, Woburn, MA (US); Daniel Arthur Wollin, Newton, MA (US); Adam Stuart Kibel, Boston, MA (US)

(21) Appl. No.: 19/066,443

(22) Filed: Feb. 28, 2025

Related U.S. Application Data
(60) Provisional application No. 63/559,538, filed on Feb. 29, 2024.

(51) Int. Cl. A61B 34/00; A61B 34/20; A61B 34/30; A61B 90/00; A61M 25/01

(52) U.S. Cl. CPC A61B 34/71; A61B 2034/2065; A61B 2034/301; A61B 2090/067; A61M 25/0113; A61M 25/0147; A61M 2025/0166

(57) ABSTRACT

The present disclosure relates to an apparatus and method for steering a continuum robot through an anatomy, including receiving an identification of a first target location; inserting the continuum robot into the anatomy; steering, during a first navigation, the continuum robot along a first path toward the first target location; storing, in a memory, location data of a plurality of points along the first path; receiving a selection of at least two points of the plurality of points; editing location data of the selected at least two points; and steering, during a second navigation, the continuum robot along a second path toward a second target location, with the second path differing from the first path based on the edit of the location data of the selected at least two points.

### CROSS-REFERENCE TO RELATED APPLICATION(S)

The present application claims priority to U.S. provisional application 63/559,538, which was filed with the on Feb. 29, 2024, the entire disclosure of which is incorporated by reference herein.

### BACKGROUND

### Field of the Disclosure

The present disclosure relates generally to medical devices and, more particularly, to methods and apparatus to navigate a continuum robot to guide interventional tools and instruments, such as endoscopes and catheters, through tortuous intraluminal paths.

### Description of Related Art

Endoscopy is a medical procedure allowing remote inspection, manipulation, and/or treatment of internal organs using flexile devices inserted through natural orifices. The complexity of anatomical pathways in a patient, the limited flexibility of existing instruments, and the need to use multiple instruments limit such procedures. Endoscopes are generally composed of a passive proximal section and an active distal section. The proximal passive section can be rigid, semi-rigid, or flexible. The active distal section includes a steerable tip that is manually controlled or remotely actuated by control wires connected to actuation wheels located on the handle of the device. Endoscopes are typically equipped with an imaging device (a camera), a light source, irrigation and/or suction channels, and at least one instrument channel for passing interventional tools. Typical endoscopic instruments have at least three degrees of freedom (3DOF) which allow for insertion, rotation and grasping operations. However, confined workspaces and limited visibility of target organs further limit the usability of these systems and require one or more highly skilled endoscopists to perform dexterous tasks. For example, four hands are required to manually control the endoscope and its instruments during a simple procedure. To that end, an endoscopist needs to master a combination of accurate tip angulations, shaft management, and instrument insertion procedures, while communicating with an endoscopist assistant to actuate the instrument and hold the endoscopic shaft in a correct position. To alleviate the complexity of such procedures, several innovative techniques have been proposed. In particular, robotic endoscope systems have been developed.

The following list of patent and non-patent publications provides some examples of manual and robotic endoscope systems. U.S. Pat. Nos. 2,975,785, 3,253,524, 3,610,231, 3,788,303, 4,207,873, 5,355,871, 8,657,781, 9,737,373, 10,470,831, US 20100041949, US 20120078053, US 20190261830, Pierre Berthet-Rayne, et al., “The i2Snake Robotic Platform for Endoscopic Surgery”, Ann Biomed Eng. 2018 October; 46(10):1663-1675, 2018; and Esther D. Rozeboom, et al., “Feasibility of joystick guided colonoscopy”, J Robotic Surg. 9:173-178, 2015.

During a manual ureteroscope procedure, a physician may insert a ureteroscope and guide the ureteroscope to a position identified as being located near a urinary stone. US 2021/0298590 to Ayvali discusses an application that records urinary stone locations, identifies target papilla, and records position(s) of target papilla. US 2004/0249267 to Gilboa discusses a system to facilitate navigation to a target within a branched structure, e.g., the bronchial tree, to guide a medical tool to the target.

Ureteroscopy for transurethral lithotripsy may be performed with a robotic ureteroscope or continuum robot that includes a bending section with flexible body. A physician may control the robotic ureteroscope by control via a joystick of the bending section to navigate through the urinary system. A type of continuum robot is discussed by U.S. Pat. No. 11,685,046 to Takagi et al. Gauhar, V., et al., Robotic Retrograde Intrarenal Surgery: A Journey from “Back to the Future”, Journal of Clinical Medicine (Vol. 11, Issue 18) 2022, discusses robotic platforms available for flexible ureteroscopy, including that a robotic device with a manual ureteroscope for recording and replaying input. Gauhar is available at https://doi.org/10.3390/jcm11185488.

US 2017/0340241 to Yamada discusses generating a bronchial image, acquiring position information of an endoscope in a bronchus, acquiring passage position information representing a passage position of the endoscope, and identifying portions through which an endoscope can or cannot be passed.

JP2014-050684A discusses generating a lumen image in which the shape of a lumen having a branch based on volume data being three-dimensional (3D) medical image data is drawn; generating a plurality of travel lines on the basis of the shape of the lumen having the branch; generating a composite image obtained by overlapping the plurality of travel lines showing a movement route of a view point in a virtual endoscope image of the lumen having the branch on the lumen image; and displaying the composite image to perform drawing which can discriminate the range of a travel line with the virtual endoscope image displayed from the range of a travel line with no virtual endoscope image displayed.

JP2005-522274A discusses, for catheter insertion through a bronchoscope working channel, securing a sheath in a set position, withdrawing a guide, and utilizing the sheath as a guide channel to guide a medical tool to a target.

Conventional systems require partial undocking to extract larger stone fragments, with undocking being cumbersome and time consuming. Recording input route data that was previously utilized to reach fragments in the urinary system and re-using such data for subsequent navigation, will delay the subsequent navigation, resulting in delayed completion of the relevant procedure.

### SUMMARY

The present disclosure relates to a control system of a continuum robot. More particularly, the present disclosure is directed to methods, systems and apparatus for a continuum robot configured with at least one independently manipulatable bendable section for advancing the robot through a passage, to provide improved navigation based on prior path data.

An aspect of the present disclosure provides a method for steering a continuum robot through an anatomy, with the method including receiving an identification of a first target location; inserting the continuum robot into the anatomy; steering, during a first navigation, the continuum robot along a first path toward the first target location; storing, in a memory, location data of a plurality of points along the first path; receiving a selection of at least two points of the plurality of points; editing location data of the selected at least two points; and steering, during a second navigation, the continuum robot along a second path toward a second target location, with the second path differing from the first path based on the edit of the location data of the selected at least two points.

Another aspect of the present disclosure provides a method for steering a continuum robot through an anatomy, the method including receiving an identification of a target location; inserting the continuum robot into the anatomy; steering, during a first navigation, the continuum robot along a first path toward the target location; at least partially withdrawing the continuum robot from the anatomy; creating a second path using information of the first path, wherein the second path differs from the first path based on an edit of at least two points of the first path; advancing the continuum robot into the anatomy; and steering, during a second navigation, the continuum robot along the second path toward the target location.

A further aspect of the present disclosure provides a method for steering a continuum robot through an anatomy, the method including inserting the continuum robot into the anatomy; steering, during a first navigation, the continuum robot along a first path from a location of the insertion toward a target location; storing, in a memory, location data of a plurality of points along the first path; displaying, to a user, the plurality of points along the first path; receiving a selection of at least two points among the displayed plurality of points; editing location data of the selected at least two points; and steering, during a second navigation, the continuum robot along a second path from the target location toward the location of the insertion, with the second path differing from the first path based on the edit of the location data of the selected at least two points.

Another aspect of the present disclosure provides a method for steering a continuum robot through an anatomy, the method including inserting the continuum robot into the anatomy; steering, during a first navigation, the continuum robot along a first path from a location of insertion toward a target location; storing, in a memory, first location data of a first plurality of points along the first path; at least partially withdrawing the continuum robot from the anatomy; steering, during a second navigation, the continuum robot along a second path toward the target location; storing, in the memory, second location data of a second plurality of points along the second path; determining a center line between the first path and the second path; at least partially withdrawing the continuum robot from the anatomy; and steering, during a third navigation, the continuum robot along a third path, wherein the third path follows the determined center line.

A further aspect of the present disclosure provides a robotic apparatus that includes a continuum robot including a plurality of bending sections, with each section of the plurality of bending sections configured to be bent by at least one wire; a driver configured to drive the at least one wire; a memory; and a controller that is configured to control a driving force of the at least one wire; control steering, during a first navigation, the continuum robot along a first path from an insertion location toward a first target location; store, in the memory, location data of a plurality of points along the first path; receive a selection of at least two points of the plurality of points; edit location data of the selected at least two points; and control steering, during a second navigation, the continuum robot along a second path toward a second target location.

A still further aspect of the present disclosure provides a robotic apparatus that includes a continuum robot including a plurality of bending sections, with each section of the plurality of bending sections configured to be bent by at least one wire; a driver configured to drive the at least one wire; a memory; and a controller that is configured to control insertion of the continuum robot into an anatomy; steer, during a first navigation, the continuum robot along a first path toward a target location; store, in the memory, location data of a plurality of points along the first path; control displaying, to a user, the plurality of points along the first path; edit the location data of at least two points selected among the plurality of points; and steer, during a second navigation, the continuum robot along a second path from the target location toward the location of the insertion, with the second path differing from the first path based on the edit of the location data of the selected at least two points.

Yet another aspect of the present disclosure provides a robotic apparatus that includes a continuum robot including a plurality of bending sections, with each section of the plurality of bending sections configured to be bent by at least one wire; a driver configured to drive the at least one wire; a memory; and a controller that is configured to control steering, during a first navigation, the continuum robot along a first path from an insertion of the continuum robot into the anatomy toward a first target location; control storing, in a memory, first location data of a first plurality of points along the first path; control at least partially withdrawing the continuum robot from the anatomy; control steering, during a second navigation, the continuum robot along a second path toward the target location; control storing, in the memory, second location data of a second plurality of points along the second path; determine a center line between the first path and the second path; control at least partially withdrawing the continuum robot from the anatomy; and control steering, during a third navigation, the continuum robot along a third path, with the third path following the determined center line.

These and other embodiments, objects, features, and advantages of the present disclosure will become apparent upon reading the following detailed description of exemplary embodiments of the present disclosure, when taken in conjunction with the appended drawings, and provided claims.

### BRIEF DESCRIPTION OF THE DRAWINGS

The accompanying drawings, which are incorporated herein and form part of the present disclosure, illustrate various embodiments, objects, features, and advantages of the present disclosure.

FIG. 1 illustrates components of a system according to an embodiment;

FIG. 2 illustrates components of data storage according to an embodiment;

FIG. 3A illustrates components of an endoscope according to an embodiment;

FIG. 3B illustrates a structure of an endoscope according to an embodiment;

FIG. 4 illustrates components for operation of an endoscope according to an embodiment;

FIG. 5 illustrates an environment of use of embodiments;

FIG. 6 illustrates a method of planning an operation utilizing a continuum robot according to an embodiment;

FIG. 7 illustrates a method for operation of continuum robot according to an embodiment;

FIG. 8 is a virtual view of first location data of ureteroscope locations according to an embodiment;

FIG. 9 is a virtual view of an insertion path and retraction path according to an embodiment;

FIG. 10 illustrates first location data according to an embodiment;

FIG. 11 illustrates first location data after edit/modification according to an embodiment;

FIG. 12 is a virtual view of navigation paths according to an embodiment;

FIG. 13 illustrates a selection method of a prior navigation history;

FIG. 14 is a virtual view of centerlines extracted in a segmented kidney model according to an embodiment;

FIG. 15 is a virtual view of locations to be removed for a subsequent navigation according to an embodiment;

FIG. 16 illustrates a method to automatically create input signals for a next navigation according to an embodiment;

FIG. 17 illustrates a method for recording input signals during retraction according to an embodiment;

FIG. 18 illustrates a method for modification of insertion path navigation according to an embodiment; and

FIG. 19 illustrates a method for generating an automated navigation path based on retraction path input signals.

Throughout the figures, similar reference numerals and characters, unless otherwise stated, are used to denote like features, elements, components, or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

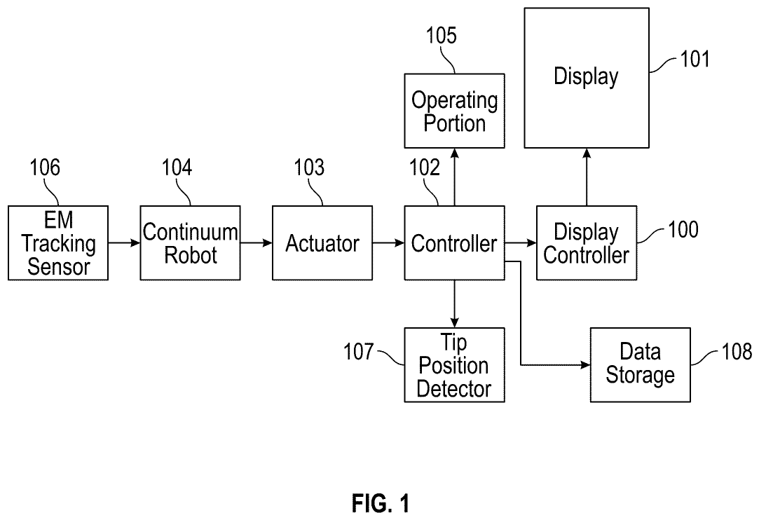

FIG. 1 illustrates components of a system according to an embodiment. Display controller 100 illustrated in FIG. 1 may execute a software program to control displaying of a navigation screen on display 101. Display controller 100 may generate a 3D model of a structure, e.g., a patient's urinary system, based on images of the patient's anatomy, e.g., computed tomography (CT) images, magnetic resonance imaging (MRI), etc. The 3D model may be received by the display controller 100 from another device. A two-dimensional (2D) model may be used instead of a 3D model. The 2D or 3D model may be generated before a navigation starts. Alternatively, the 2D or 3D model may be generated in real-time, e.g., parallel with the navigation. An example of generating a model of urinary system is provided, without limiting the present disclosure to modeling the urinary system. For example, a model of a route direct to a target may be used instead of the urinary system. Alternatively, a model of a broad space may be used. The model may represent a place or a space where an observation or a work is performed using a continuum robot 104, alternatively referred to an endoscope or ureteroscope herein.

The display controller 100 may interface with controller 102 to acquire position information of endoscope 104. The endoscope 104 may include an EM tracking sensor 106 on a distal end thereof. The display controller 100 may also acquire the position information directory from a catheter tip position detector 107, via the EM tracking sensor 106. That is, during the first navigation disclosed herein, first location data is obtained from the EM tracking sensor 106.

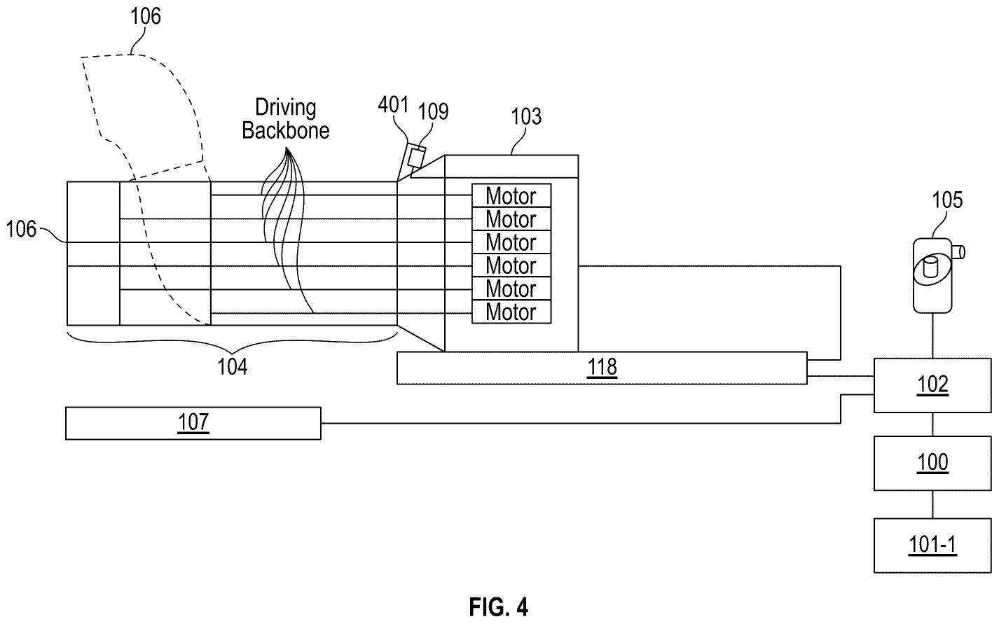

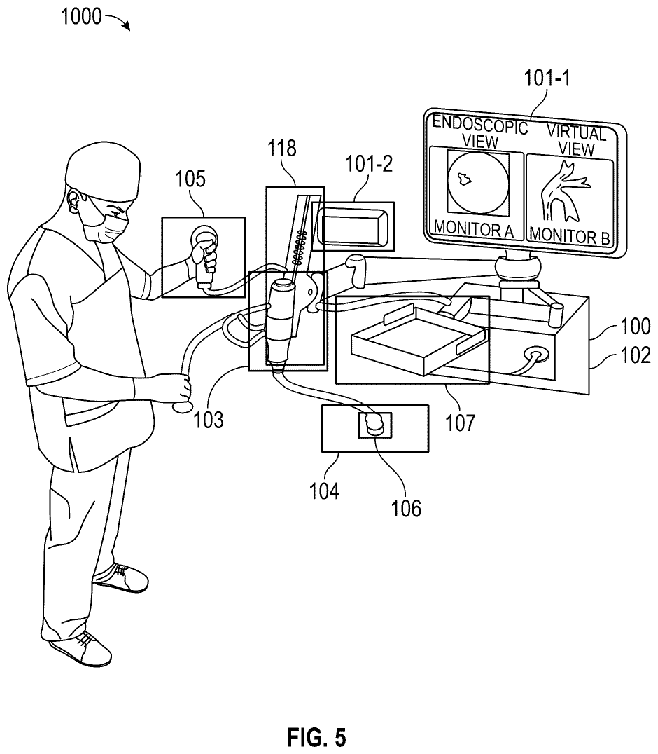

The controller 102 may receive information regarding a location of a tip of the endoscope 104, referred to as positional information, from the catheter tip position detector 107. The positional information may be stored in data storage 108. The controller 102 may send input signals to control an actuator 103 and a rail 118 based on user manipulation via one or more operation portions (FIGS. 4 and 5). The input signals are recorded and stored in the data storage 108. In an embodiment, the one or more displays 101-1, 101-2, and/or handheld controller 105, e.g., joystick, provide a user interface. In other embodiments, the user interface includes voice control.

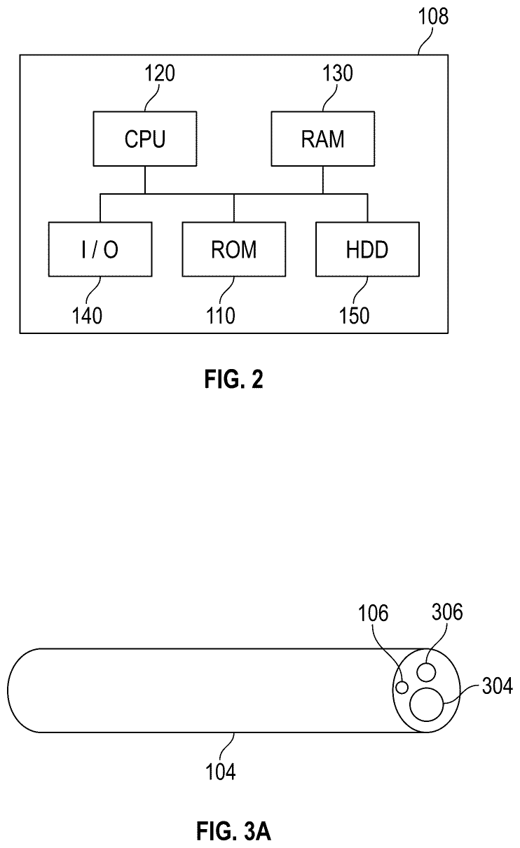

FIG. 2 illustrates components of the data storage 108 according to an embodiment. As illustrated in FIG. 2, the data storage 108, i.e., memory, may store images of a segmented kidney model created from a preoperative CT scan. The display controller 100 and controller 102 may include at least one read only memory (ROM) 110, at least one central processing unit (CPU) 120, at least one random access memory (RAM) 130, at least one input and output (I/O) interface 140, and at least one hard disc drive (HDD) 150. A solid-state drive (SSD) may be used with or instead of the HDD 150. The data storage 108 may be provided as part of display controller 100 and as part of controller 102. The display controller 100 may be provided as part of controller 102.

The ROM 110 and/or HDD 150 may be used to store software programs, and the RAM 130 may be used as a working memory. The CPU 120 may be used to execute one or more software programs stored in the RAM 130. The I/O 140 may receive positional information for input to the display controller 100 and may output information for displaying the navigation screen to the one or more displays 101-1 and/or 101-2, as illustrated in FIGS. 4 and 5. The navigation screen may be generated by the software program but may be generated by firmware.

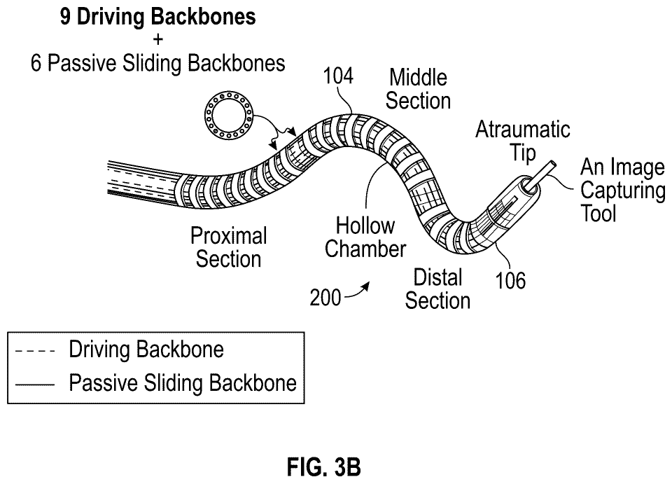

FIG. 3A illustrates components that may be provided within an endoscope 104 according to an embodiment.

As shown in FIG. 3A, the endoscope may be a scope device that is attachable/detachable to the actuator 103. The endoscope 104 may be disposable. For conciseness, the description herein references the endoscope 104 as being a ureteroscope. However, the endoscope 104 is not limited to a ureteroscope and may include endoscopic devices used for other organs. That is, as used herein, the term endoscope refers to a rigid, or flexible medical instrument which uses light guided by an optical probe to look inside a body cavity or organ, that is steerable. The endoscope may include, or be configured to send to a processor, information regarding the location and/or movement of the endoscope, thus providing data on the path taken during a procedure. A medical procedure, in which an endoscope is inserted through a natural opening, is called an endoscopy. The inserting and steering may be manual operations that are performed by a user/physician, may be computer controlled, or may be a combination thereof.

Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes. Embodiment(s) of the present disclosure can also be realized by a computer of a system or apparatus that reads out and executes computer executable instructions (e.g., one or more programs) recorded on a storage medium (which may also be referred to more fully as a non-transitory computer-readable storage medium) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., a CPU, micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a RAM, a ROM, a storage of distributed computing systems, an optical disk (such as a compact disc (CD), digital versatile disc (DVD), or Blu-ray Disc (BD)™), a flash memory device, a memory card, and the like. An I/O interface can be used to provide communication interfaces to input and output devices, which may include a keyboard, a display, a mouse, a touch screen, touchless interface (e.g., a gesture recognition device) a printing device, a light pen, an optical storage device, a scanner, a microphone, a camera, a drive, communication cable and a network (either wired or wireless).

The endoscope 104 may, but need not necessarily, include one or more cameras or imaging devices 306. The endoscope 104 may be a catheter that does not include a camera or other imaging device 306. As illustrated in FIG. 3A, the catheter may include a tool channel 304. A camera may be inserted through the tool channel 304 or a separate imaging capturing unit 306 may be provided on a distal end thereof. The tool channel 304 is configured to receive medical tools, e.g., an EM tracking sensor 106, a laser, forceps, a needle or other biopsy tools, a basket catheter, etc., without limitation thereto. The tool is inserted or removed through a tool insertion slot 401, as illustrated in FIG. 4.

FIG. 3B illustrates a structure of an endoscope according to an embodiment. As illustrated, the endoscope 104 may include a proximal section, a middle section, and a distal section, and each section may be bent by operation of a plurality of driving wires, i.e., driving liner members, with the posture of the endoscope 104 being supported by supporting wires, i.e., supporting liner members. The plurality of driving wires are connected to the actuator 103, which includes one or more motors configured to drive sections of the endoscope 104 by pushing and/or pulling the driving wires. In some embodiments, a rail 118 is included, where the actuator 103 proceeds or retreats along the rail 118, and the actuator 103 and endoscope 104 may proceed into or retreat from within the patient's body. An electro-magnetic (EM) tracking sensor 106 may be attached to the tip of the endoscope 104. In certain embodiments, a robot 104 may include the EM tracking sensor 106 on a distal end thereof. The image capturing tool, e.g., imaging device 306, may be inserted through the endoscope 104.

FIG. 4 illustrates configurations of an endoscope according to an embodiment. The catheter tip position detector 107 detects a position of the EM tracking sensor 106 and outputs the detected positional information to the controller 102. In response to user input via the operating portion, e.g., handheld controller 105 and/or one or more of displays 101-1, 101-2, the actuator 103 may move along the rail 118 and the actuator 103 may operate certain driving wires of the plurality of driving wires to change a position of the distal end of the endoscope 104 and the EM tracking sensor 106.

FIG. 5 illustrates an environment of use of an embodiment. The display controller 100 generates and outputs data to provide the navigation screen on one or more of display 101-1 and 101-2 based on the 3D model and the position information, based on executed software. The navigation screen displays a current position of the endoscope 104 on the 3D model. Based on information displayed on the navigation screen, a user may recognize the current position of the endoscope 104 in an anatomy, hollow organ, or branched structure within the human body. As illustrated, a display 101-1 is provided as a large screen user interface with a touch panel that is configured for use as a first user interface unit. A second user interface unit is provided as display 101-2 with a touch panel. An operating portion may also be provided as a handheld controller 105 having shift lever(s)/button(s). A user can operate the system by input provided to the operating portion, e.g., one or more of display 101-1, display 101-2, and handheld controller 105.

The controller 102 may control the endoscope 104 based on an algorithm known as follow the leader (FTL) algorithm, for example as discussed in U.S. Pat. No. 11,051,892 to Hata et al., U.S. Pat. No. 11,103,992 to Tanaka et al., and US 2023/0131269 to Takagi. Also see U.S. Pat. No. 10,149,607 to Choset et al. By applying the FTL algorithm, the middle section, and the proximal section (following sections) of the endoscope 104 moves at a first position in the same way as the distal section moved at the first position or a second position near the first position, allowing the middle (and proximal section, if more than two sections) to follow the route taken by the distal section.

The controller 102 and the display controller 100 can be configured separately. The controller 102 may include a CPU, RAM, I/O, ROM and HDD, as shown in FIG. 2. Alternatively, the controller 102 and the display controller 100 can be configured as one device.

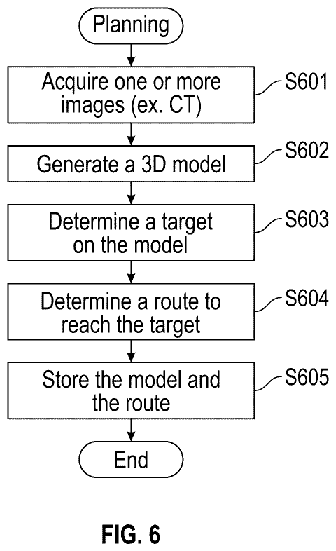

FIG. 6 illustrates a method of planning an operation utilizing a continuum robot according to an embodiment. The steps illustrated in FIG. 6 may be performed by the by CPU 120 executing a software program stored in ROM 110 or HDD 150.

In step 601 of FIG. 6, images such as CT or MRI images are acquired. A 3D model of an anatomical structure, e.g., a urinary system, is generated in step 602 based on the acquired images. Based on user instruction, a target in the anatomical structure is determined in step 603. Optionally, in step 604, a route for the endoscope 104 to travel to the target is determined. The generated three-dimensional model, with optionally determined route, is stored in the RAM 130 or HDD 150 in step 605. Thus, a 3D model of the anatomical structure is generated, and a target on the 3D model with optional route is determined and stored before the operation of the endoscope 104 is started.

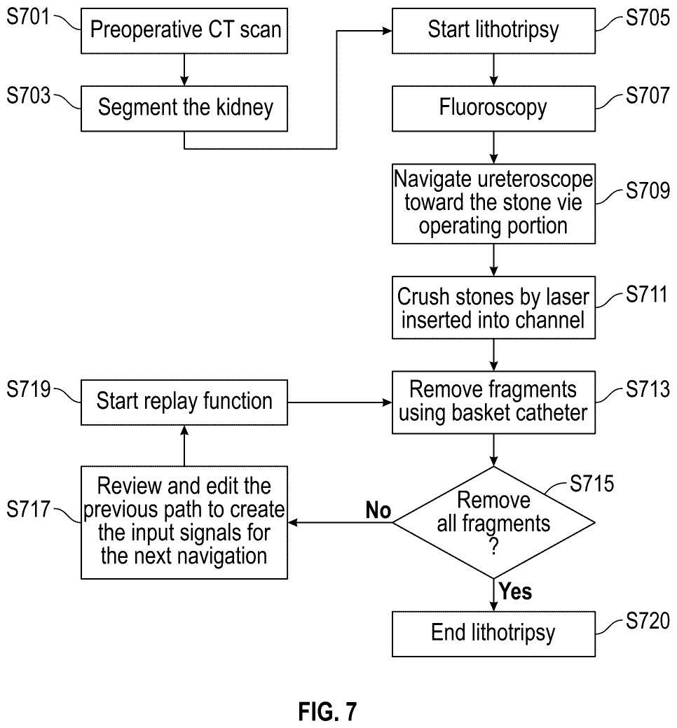

FIG. 7 illustrates a method for operation of continuum robot according to an embodiment.

Preoperative CT imaging of a hollow organ, e.g., a kidney, is performed in step S701. The preoperative CT imaging scan need not be performed on the same day as the lithotripsy procedure. The inner wall of the kidney is segmented in step S703 and the segmented kidney model is stored in data storage 108. To break up the urinary stone, a lithotripsy procedure commences in step S705.

At the start of a lithotripsy procedure, in step S707, a physician obtains a fluoroscopic image to confirm the location of the urinary stone. The physician then checks the segmented kidney model displayed in a virtual view on a display 101.

In step S709 the physician navigates the ureteroscope to the urinary stone. After inserting the ureteroscope into the ureter, the physician may insert an access sheath through the urinary tract, to the renal pelvis.

Point-set registration is performed to obtain the transform between the patient coordinate frame and a coordinate frame of an EM tracking system that is in communication with the EM tracking sensor 106. That is, using the EM tracking sensor 106 that is attached to the ureteroscope 104, the physician may navigate the ureteroscope 104 to landmarks or a target in the urinary collecting system. After completing the point-set registration process, a real time location of the ureteroscope 104 may be displayed in the virtual view on display 101 in the segmented kidney model.

In an initial navigation, the physician inserts the ureteroscope 104 into the urinary collecting system and navigates the ureteroscope 104 via input to the operating portion, e.g., handheld controller 105 and/or one or more of displays 101-1, 101-2. The physician navigates to the urinary stone that is displayed in the fluoroscopic image. The physician inserts the ureteroscope 104 into the access sheath, and stops at the exit of the access sheath. While navigating from the exit of the access sheath to the urinary stone, the EM tracking system sends a real-time location data of each position of the ureteroscope 104 to the controller 102. The real-time location data may be recorded in the data storage 108 at designated intervals, e.g., every 100 msec, with the input signals sent by the physician to the controller 102 to control the actuator 103 and the rail 118.

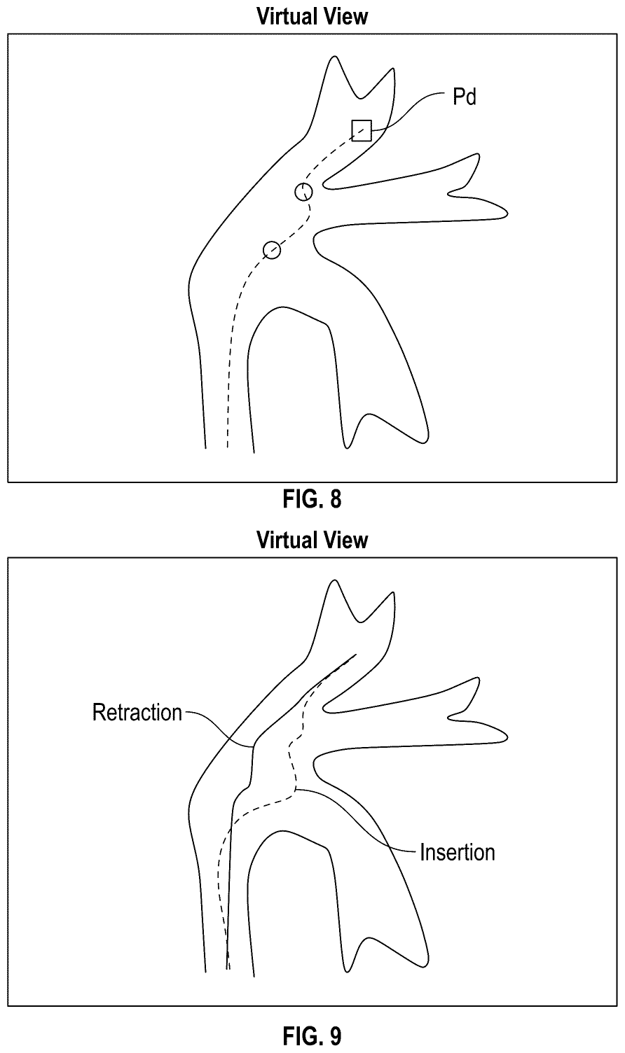

FIG. 8 is a virtual view of first location data of ureteroscope locations according to an embodiment. As illustrated in FIG. 8, a virtual view the first location data may be output to a display 101, and may include a location of the ureteroscope 104. The location information may be stored in data storage 108 for a first plurality of points along the first path, including at least one of time stamp data, location data, bending angle data, bending plane data, and rail position data.

Once the ureteroscope 104 reaches the urinary stone, a laser fiber may be inserted through the tool channel 304 and the physician may crush the stone into fragments in step S711 of FIG. 7. In step S713, the physician retracts the laser fiber and deploys a basket catheter through the tool channel 304, to catch the fragments. Once the basket catheter catches a fragment, the physician retracts the ureteroscope 104 with the basket catheter so that the fragment can be discarded outside of the patient's body.

FIG. 9 is a virtual view of an insertion path and retraction path according to an embodiment.

After removing the ureteroscope 104 via the retraction path, in step S715 of FIG. 7, the physician reviews whether all fragments have been removed. If the physician determines that all fragments have been removed, the lithotripsy ends at step S720. If the physician determines that all fragments have not been removed, the ureteroscope 104 and the basket catheter must be reinserted. Thus, in step S717, a review is performed of first path data obtained during the initial navigation, with the first path data including one or more of first path data obtained during insertion of the ureteroscope 104 or first path data obtained during removal of the ureteroscope 104.

FIG. 9 illustrates output of a method for steering a continuum robot that includes performing a first insertion of the continuum robot into a hollow organ. After the insertion, steering is performed during a first navigation for the continuum robot to travel along a first path from a location of the first insertion toward a first target location. First location data of a first plurality of points along the first path is stored in a memory, i.e., data storage 108, and the first plurality of points along the first path are displayed to a user. The first location data of at least two points selected among the first plurality of points is then edited and a second navigation commences. During the second navigation, the continuum robot is steered or guided along a second path from the first target location toward the location of the first insertion. As illustrated in FIG. 9, a location of the first insertion is substantially the same as a location of the second insertion, and the second path is shorter in length than the first path. The second path differs from the first path based on modification/edit of the first location data of the at least two selected points. This modification may be made, for example, since the first insertion required additional movement for the clinician to observe the calyx, search for stone fragment(s), etc. In later insertions, the clinician may be interested in moving to substantially the same location of the first insertion without the need to look around and/or search for a stone fragment. The insertion may through any anatomy accessible to an endoscope, such as in a hollow organ (e.g., kidney), an organ having a branched structure (e.g., lung), or through tissue (e.g., brain).

Points may be displayed for selection for a diversion, i.e., modification/edit. For example, as illustrated in FIG. 8, a first plurality of points may be displayed as a virtual map of the first path. See, e.g., the curved path between points A and B along the first path for such diversion created by edit of the first path, of at least two points of the displayed first plurality of points for the modification/edit. In some embodiment, the point selection and/or diversion may be defined by the user. In other embodiments, the processor selects the points for modification.

Thus, modification/edit of the historical path data obtained during the initial or first navigation may be performed to determine second path data for a second insertion of the ureteroscope 104, with the controller 102 determining the second path data by editing the stored first path or by editing the removal path of the continuum robot from the object.

Display in the virtual view allows the physician to readily identify at least two points along the first path that are unnecessary to reach the target. The closer in time and/or location that the two points are to each other, a reduced risk exists of unpredictable behavior that the ureteroscope might encounter if the physician elects to replace the diversion between the two points with a straight path, i.e., edit the path. In an embodiment, a distance of a part of the path that the user can specify as being a diversion or as being unnecessary may be limited, with the distance being a direct distance between the two points that the user designates. Alternatively, the distance may be a distance along the path between the two points. The controller 102 may be configured to accept from the user the designations of the two points within the limited distance of the part of the path or a limited distance therebetween.

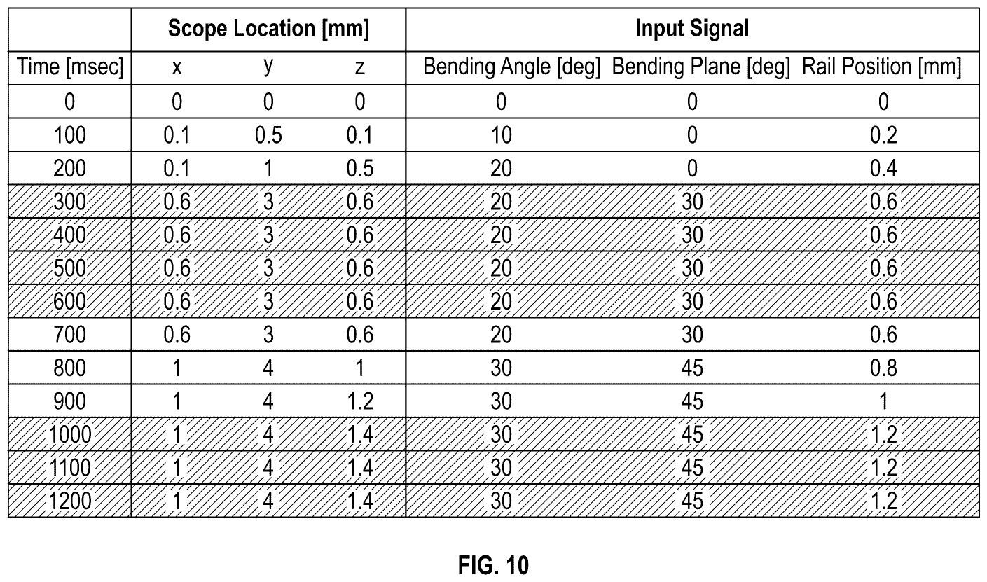

FIG. 10 illustrates first path data according to an embodiment. The table in FIG. 10 illustrates data stored in memory 108 during the first navigation.

In an embodiment, an edit time stamp is utilized to remove an unnecessary part of a path. Controller 102 or display controller 100 may remove the unnecessary part of the path by editing a time stamp associated with positional information of the path.

Data storage 108 stores at least one of bending angle information and bending plane information for each point the first plurality of points, thus allowing the second path to be created by modification/edit of the stored at least one of the bending angle information and the bending plane information for each point of the selected at least two points of the first path.

In FIG. 10, the scope location header indicates positional information of the catheter tip position sensor 107 at each time interval while the ureteroscope is steered along a path. The input signal header indicates a bending angle and bending plane of the bendable portion of the ureteroscope inputted to the controller 102 at each time interval while the ureteroscope is steered. The input signal also indicates a position of the rail 118. The ureteroscope 104 and the actuator 103 are supported on the rail 118. When the ureteroscope 104 and the actuator 103 advance along with the rail 118, the ureteroscope 104 advances deeper into the human body.

A physician may pause advancement of the ureteroscope 104 to consider a direction in which to move during the first navigation. During the pause, the controller 102 may continue to record the real-time location of the ureteroscope 104 and the input signals that are sent to the controller 102 to control the actuator 103 and the rail 118 at designated intervals, e.g., every 100 msec, during the pause. In an embodiment, the pause is eliminated from the second navigation. Thus, the display controller 100 edits the input signal so that the redundant input signals are deleted, to generate input signal for the second navigation.

That is, based on stored time stamp data, redundant locations where the continuum robot is stationary are determined, and cumulative location data where the continuum robot is determined to be stationary are either deleted or not stored.

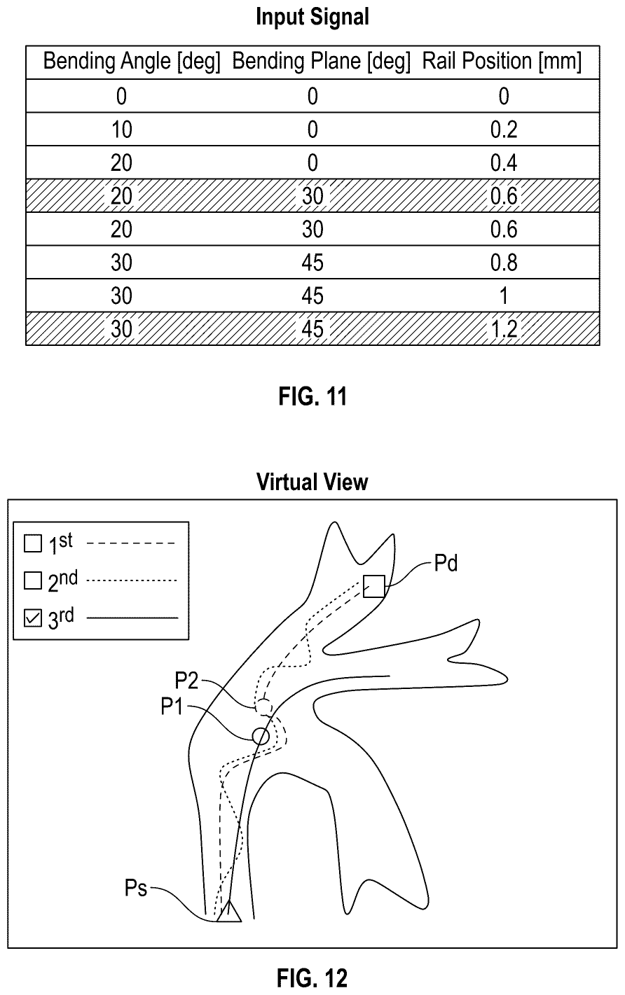

FIG. 11 illustrates first location data after edit/modification according to an embodiment. The table in FIG. 11 shows the input signals to be sent to controller 102 for the second navigation, i.e., navigation along the second path. As illustrated in FIG. 11, the second path data omits input signals between time periods of 400 msec to 600 msec and 1100 msec to 1200 msec that are included in the first path data in FIG. 10.

That is, after the first navigation, i.e., navigation along the first path, the display controller 100 detects whether the physician takes a pause based on the input signals recorded during the first navigation. If the signals of the scope location and the input signal are identical during consecutive times, as illustrated in the rows having gray color in FIG. 10, the display controller 100 determines that a pause exists during the period. For example, if the scope location, bending angle, bending plane, and rail position are the same for more than a predetermined time period, e.g., 200 msec or 300 msec, the display controller 100 may edit the input signal during the period.

To prevent the ureteroscope 104 from repeating the first navigation pause during the second navigation, i.e., navigation along the second path, the display controller 100 may merge duplicated input signals into a single input signal to be used during the second navigation. For example, FIG. 11 omits rows of 400 msec, 500 msec and 600 msec that appear in FIG. 10, as well as rows of 1100 and 1200 msec. The display controller 100 may then allocate a consecutive time stamp to each row in 100 msec increments.

For conciseness, the above discussion only edits same signals. However, the signals can be edited if the consecutive x coordinates are the same, even if the y and z coordinates are not the same. Alternatively, the signals can be edited if the consecutive rail positions are the same, even if the bending angle and the bending plane are not the same. Alternatively, the display controller 100 may record the real-time location of the ureteroscope and the input signals sent to the controller to control the actuator 103 and the rail 118 when the display controller 100 sends the input signals to the actuator 103 and the rail 118, to remove duplicated input signals from the second navigation.

Controller 102 or display controller 100 may copy the input signals of the first path data that were previously sent to the controller 102 during the first navigation for edit/modification thereof, to create input signals to use in a process to create second path data for the second navigation. The controller 102 may delete the input signals between the two points identified as the unnecessary part of the path. The controller 102 interpolates the input signals for the second insertion so that the interpolated second path, like the first path, is within the lumen of the kidney.

Thus, a method is provided for steering a continuum robot through the lumen or hollow of the kidney or other organ that includes receiving an identification of a first target location and performing a first insertion of the continuum robot into the organ. During a first navigation, the continuum robot is steered/guided along a first path from the first insertion toward the first target location and first location data of a first plurality of points along the first path is storing in data storage 108. The first plurality of points along the first path may be displayed to a user and a second insertion of the continuum robot may be performed. During a second navigation, the continuum robot is steered/guided along a second path from the second insertion toward a second target location. The second target location may be, for example, at substantially the same location as the first target location, in a different location, but in the same calyx, in a different calyx, a point proximal to the first target location along the path. The first location data of at least two points selected among the first plurality of points is edited/modified, and the second path will differ from the first path based on the edit of the first location data of the at least two selected points. In an embodiment, a location of the first insertion is substantially the same as a location of the second insertion. Also, the editing/modifying of the first location data of the at least two selected points may include editing/modifying stored time stamp data for each of the at least two selected points. Data of points located between the selected at least two points of the first path being interpolated to create the second path.

Thus, the first path data is revised to exclude unnecessary diversions between points A and B, as illustrated in FIG. 8, resulting in a more time efficient path for the ureteroscope 104 to follow when the ureteroscope 104 with the basket catheter are reinserted at step S719 to follow the second path to catch remaining fragments in step S713. The display controller 100 may again accept the identification of the two points after the interpolation is performed. If so, the display controller 100 repeats the replacement of the unnecessary path with a further interpolated path.

FIG. 12 is a virtual view of navigation paths according to an embodiment. The second path illustrated in FIG. 12 has fewer diversions than the second path, and the third path has fewer diversions than the first path. Thus, the physician can save additional time during a third navigation by eliminating redundancies from the first path data and the second path data.

In the virtual view illustrated in FIG. 12, the physician may set one point as a destination point P_{d} of a next navigation. Thus, a collected path of the ureteroscope 104 is obtained based on the first path data. In step S719 of FIG. 7, the physician may start second navigation for removal of remaining fragments utilizing a modified replay function of at least one prior path data. The physician inserts the ureteroscope 104 into the urinary system through the access sheath, and stops at the exit of the access sheath. Then, the physician starts the modified replay function to commence a next navigation. The display controller 100 sends information of modified path data to the controller 102. Use of the modified path data in the replay function causes the controller 102 to send input signals for the second insertion to control the actuator 103 and the rail 118 in accordance with the modified path. The insertion stops when the ureteroscope reaches the destination P_{d} or when the physician takes manual control. That is, during the modified replay function, the user may stop automatic steering of the ureteroscope in accordance with the modified path in the middle of the path to the destination P_{d}, thus allowing the user to manually steer the ureteroscope.

During navigation from the exit of the access sheath to the destination, the EM tracking system sends real-time location data of the ureteroscope 104 to the controller 102. The real-time location data of the ureteroscope 104 may be recorded in the data storage 108 at designated intervals, e.g., at every 100 msec, with the input signals sent to the controller 102 to control the actuator 103 and the rail 118. The physician deploys the basket catheter through the tool channel 304 to catch the fragments. Once the basket catheter catches some of the fragments, the physician retracts the ureteroscope 104 with the basket catheter to the outside of the patient's body.

After a second removal process of fragments, the physician may review and edit the input history to create the input signals for a third insertion. The physician's review may be of the collected path of the ureteroscope of the second navigation in the virtual view, to see if locations that are virtually displayed include unnecessary locations. Utilizing the operating portion, the physician may identify an unnecessary part of the path by selecting at least two points among the plurality of points that are virtually displayed.

In certain embodiments, the target may move. Thus, the second path of the second navigation may contain additional at least one unnecessary part of the path for the third navigation, despite the second navigation having used a second path that was modified from the first path. In addition, the replay function allows the user to stop automatic steering of the ureteroscope along the modified path in the middle of the path to the destination, thereby allowing the user to steer the ureteroscope manually. Such stop of automatic steering is necessary due to the movement of fragments that may occur due to the flow of the urine and/or organ motion. When the physician finds fragments before the ureteroscope reaches the target destination, the physician can stop the replay function and remove the relocated fragment by operation of the operating portion.

Thus, the history of the path may contain unnecessary parts caused by a physician having paused to search for a urinary stone inside the kidney using the ureteroscope or by ureteroscope movement during such physician search. Such movement of the ureteroscope 104 adds unnecessary and potentially redundant path data.

To create input signals for the third navigation, the controller may copy the input signals that were sent to the controller during the second navigation. The controller may then delete the input signals that are between the two points identified as the unnecessary part of the path. The controller interpolates the input signals to create the third path. In the virtual view, the physician may then set one of the two points as a destination of the next navigation. The copying and editing of signals used for the prior navigation is repeated until the physician removes as many fragments as possible.

In some embodiment, where the second navigation to the second target point is to a point substantially nearby or close to (e.g., within 30 mm, 20 mm, 10 mm or less) the first target point, advantages of the apparatus and methods as described herein can be particularly advantageous, particularly on the second and later navigations since they can provide increased navigation speed, increased confidence of returning to the target location; improved automation; and reduced user fatigue.

In an embodiment, the controller 102 may be configured to only run the replay function when the physician holds, on the handheld controller 105, a button for the replay function. Upon release of the button in the middle of navigation, the physician can manually navigate the ureteroscope 104 using the operating portion.

In an embodiment, a part of a first path and a part of a second path are used to generate a path for a next navigation, with the first path data and second path data providing histories of respective previous navigations.

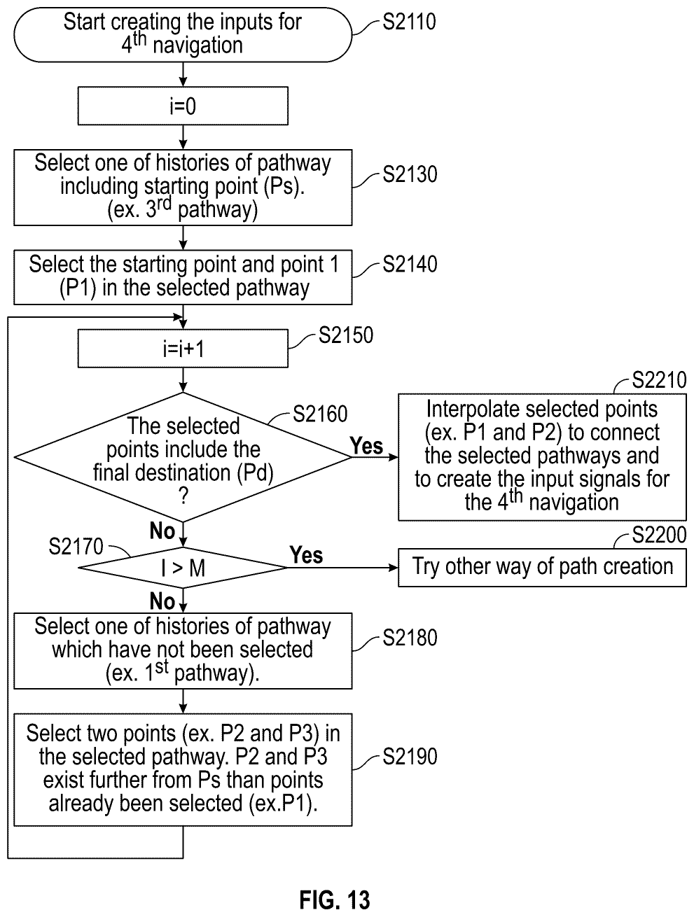

FIG. 13 illustrates a selection method of a prior navigation history.

In step S2110 of FIG. 13, a user initiates creation of inputs for a fourth navigation, as an example of a route navigation that is to be automatically performed. In step S2120, a count of selected pathway, e.g., variable “i” is set to 0. In step S2130, the display controller 100 selects one of the histories of a pathway, including a starting point (Ps). For example, the display controller 100 selects the third pathway, as illustrated in FIG. 12, with the starting point Ps and destination point Pd being designated by a user.

In step S2140, based on the selected starting point (Ps), the display controller 100 may select a starting point and point 1 (P1) in the selected pathway. For example, a closest point to the destination point Pd on the selected path may be selected as P1. In step 2150, i is incremented, e.g., i+1.

In step S2160, the display controller 100 may determine whether the selected points include the final destination point Pd. If so, in step S2160, the display controller 100 may interpolate data points from previous navigation histories to connect the selected points to generate the new fourth navigation path in step S2210.

If in step 2160, the display controller 100 does not determine that the selected points includes the final destination point Pd, in step S2170 the display controller 100 determines whether i is less than a threshold M, with M being a maximum limit of the number of paths that are to be combined. If i equals the M, in S2220 the display controller 100 ends the process and outputs a user prompt to create a different path, e.g., manually select a path by manual designation of connection points.

If in step 2170, the display controller 100 determines that i is less than the threshold M, in step S180 the display controller 100 may select at least one history of a pathway that was not selected. For example, the display controller 100 may select the first pathway in FIG. 12 and, in step S2190, may select two points (ex. P2 and P3) in the selected pathway. For example, P2 is closest on the pathway to P1, as selected in step 2140, and P3 is on the pathway to Pd. The display controller 100 may then return to step S2150 and repeat steps S2150 to S2190. In an embodiment, each step of FIG. 13 may be performed by the display controller 100, or by controller 102, by executing the software stored in the data storage 108. The histories of the previous navigation selected in FIG. 13 may be combined and automatically performed.

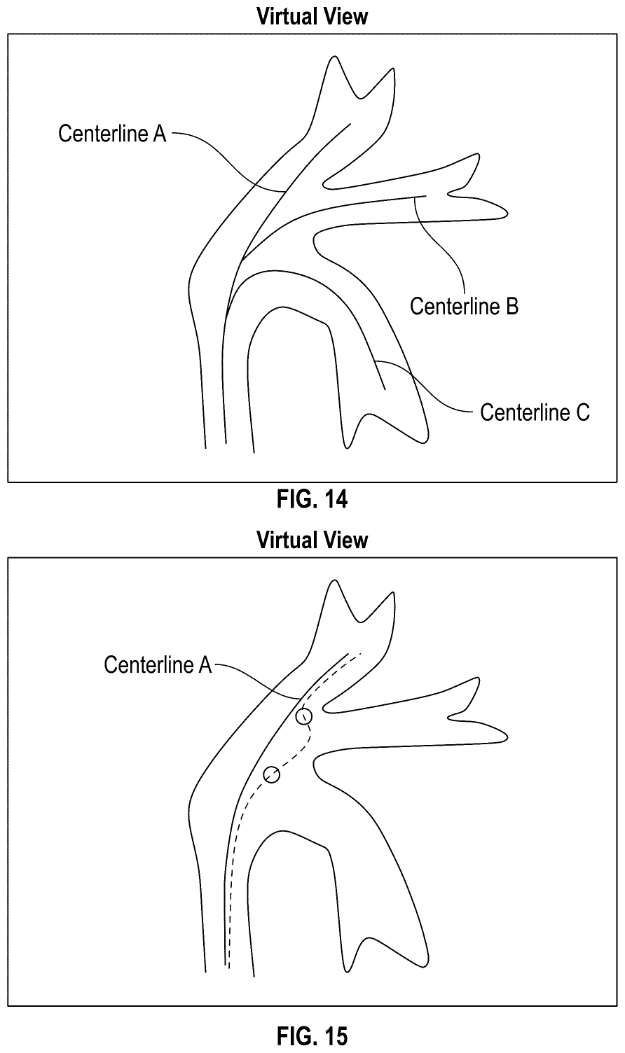

FIG. 14 is a virtual view of centerlines extracted in a segmented kidney model according to an embodiment. The three centerlines illustrated in FIG. 14 were extracted toward each calyx of a subject kidney.

FIG. 15 is a virtual view of locations to be removed for a subsequent navigation according to an embodiment.

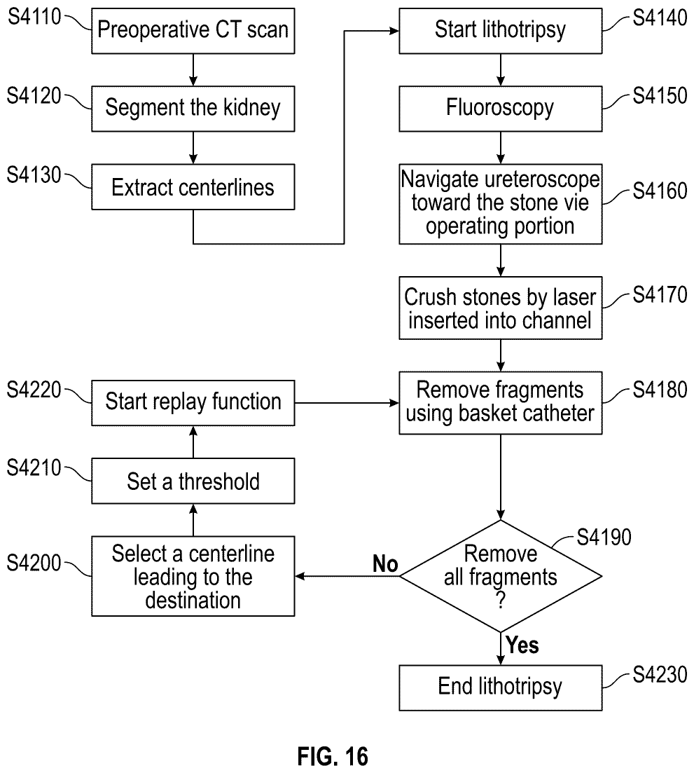

FIG. 16 illustrates a method to automatically create input signals for a next navigation according to an embodiment.

Other than steps S4130, S4210 and S4200, the steps of FIG. 16 correspond to the steps in FIG. 7. For conciseness, a description of the duplicated steps is incorporated herein by reference here, without repetition thereof. In S4130 of FIG. 16, centerlines are extracted of a segmented kidney. After removal of the fragments, at step S4200 of FIG. 16, a centerline is selected leading to a target destination. Once a first removal of the fragments is completed, the physician may select a centerline leading to the fragments to be removed and, in step S4200, may set a threshold to detect unnecessary locations for second navigation. The display controller shows the selected centerline, the history of the first navigation, and locations to be removed for the second navigation in the virtual view. The replay function then commences in step S4220, as described in step S719 of FIG. 7. According to the embodiment, the physician avoids wasting time creating input signals to be used for the next navigation.

Thus, a method is provided for steering a continuum robot through an organ or branched structure that includes performing a first insertion of the continuum robot into the organ; steering, during a first navigation, the continuum robot along a first path from the first insertion to a first target location; storing, in a memory, first location data of a first plurality of points along the first path; performing a second insertion of the continuum robot into the organ; steering, during a second navigation, the continuum robot along a second path from the second insertion toward a second target location; storing, in the memory, second location data of a second plurality of points along the second path; determining a center line between the first path and the second path; performing a third insertion of the continuum robot; and steering, during a third navigation, the continuum robot along a third path. The third path follows the determined center line and the third navigation steering is an automated replay function based on an edit of the first location data of at least two points selected among the first plurality of points and an edit of the second location data of at least two points selected among the second plurality of points. Manual control of the continuum robot may be obtained by pausing the automated replay function. Locations of the first insertion, the second insertion and the third insertion are substantially the same, and the first target location and the second target location are within the organ.

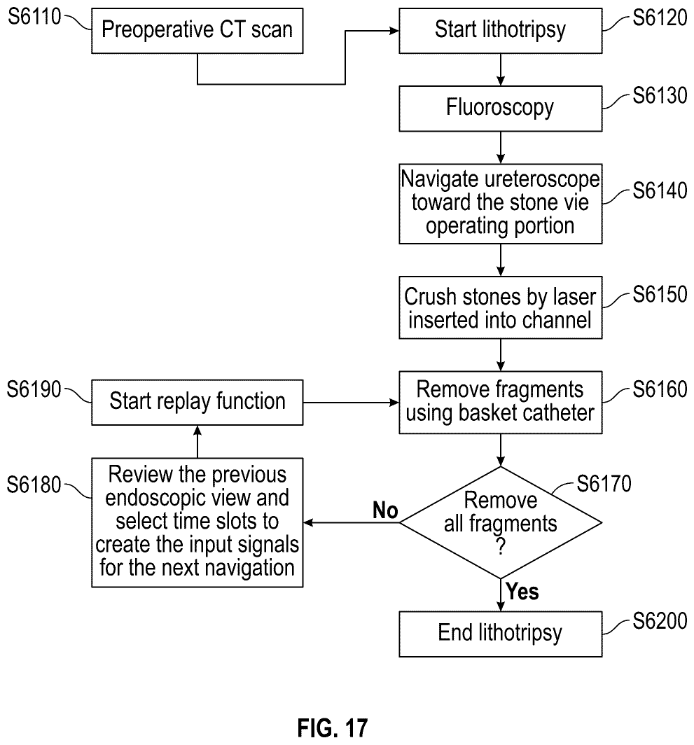

FIG. 17 illustrates a method for recording input signals during retraction according to an embodiment.

The method of FIG. 17 may be performed without use of EM tracking sensor 106, with the physician inserting an ureteroscope 104 into the urinary system and navigating the ureteroscope 104 using the operating portion toward the urinary stone shown in the fluoroscopic image obtained in step S6130, which is the same as step S707 of FIG. 7. Other than step S6180, the steps of FIG. 16 correspond to the steps in FIG. 7. For conciseness, a description of the duplicated steps is incorporated here, without repetition thereof.

During the navigation, endoscopic video is recorded at designated intervals, e.g. every 100 msec, with the endoscopic video sent to the controller 102 for control of the actuator 103 and the rail 118. In S6180 of FIG. 17, a previous endoscopic view is reviewed and time slots are created for input signals for a next navigation. The replay function then commences in step S6190, as described in step S719 of FIG. 7. Once the first removal of the fragments is completed, a physician replays the recorded endoscopic video and select time slots to be used to create the input signals for the next navigation. Thus, the physician does not have to use an EM tracking system to create input signals to be used for the next navigation.

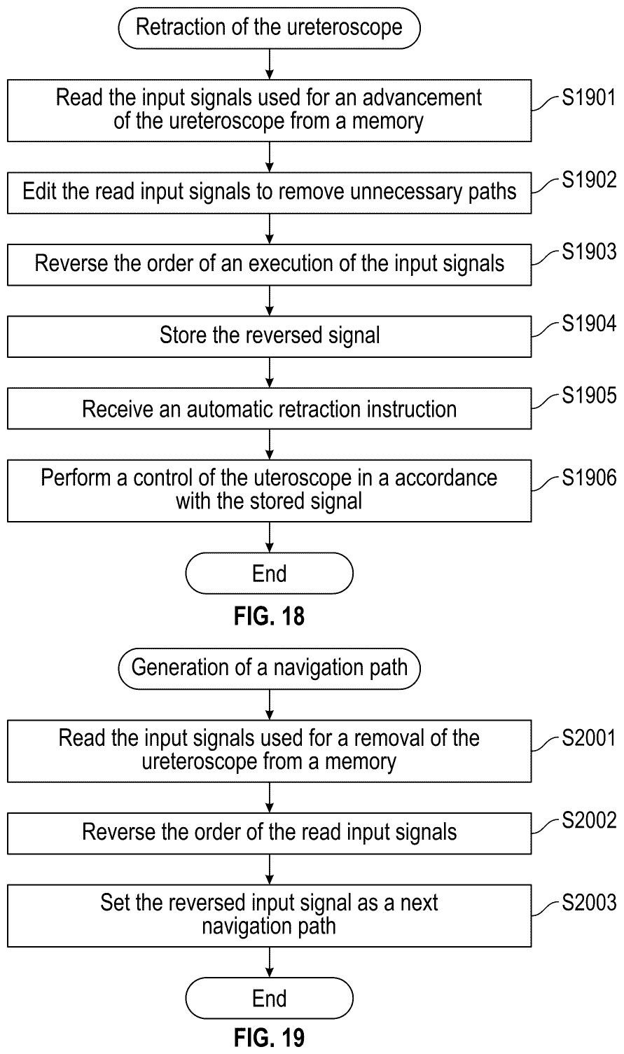

FIG. 18 illustrates a method for modification of insertion path navigation according to an embodiment. In step 1901 controller 102 reads from memory 108 input signals used to steer the ureteroscope 104. As described above, FIG. 10 shows the table of the input signals used to steer the ureteroscope to the destination, and the input signals may include scope location and input signal.

In step S1902, the controller 102 edits the input signals to remove unnecessary path data, as described above. As illustrated in FIG. 9, an insertion navigation path may include unnecessary parts caused by operational diversions while the physician initially explores the hollow or branched structure of the organ. Accordingly, the removal navigation path illustrated in FIG. 9 omits such diversions, which are not necessary during a return toward the target location. Thus, in step S1902, the controller 102 edits the input signals to remove unnecessary path data. In step S1903, the controller 102 reverses the order of the input signals and, in step S1904, stores the reversed input signals in memory 108. For example, time stamp data is edited so that the order of execution is reversed. For example, time stamp data of 0 to 700 may be edited to 700 to 0.

In step S1905, an instruction to perform automatic retraction is received and, in step S1906, automatic removal of the ureteroscope 104 is performed based on the reversed input signals that were stored in the memory 108 in step S1904.

FIG. 19 illustrates a method for generating an automated navigation path based on retraction path input signals. In step S2001 of FIG. 19, the controller 102 retrieves the input signals stored in memory 108 in step S1904 of FIG. 18. In step S2002, the controller 102 reverses the order of the input signals that were retrieved from memory 108. That is, time stamp data is edited to reverse the order of execution. For example, time stamp data of 0 to 700 may be edited to 700 to 0. In step S2003, the controller 101 sets a next navigation path using the signal that was reversed in step S2002. According to various embodiments, input signals are created for selected time slots and physician avoids wasting time creating input signals to be used for the next navigation.

Thus, a robotic apparatus is provided that includes a continuum robot including a plurality of bending sections, with each section of the plurality of bending sections configured to be bent by at least one wire; a driver configured to drive the at least one wire; a memory; and a controller that is configured to control a driving force of the at least one wire; control steering, during a first navigation, the continuum robot along a first path from an insertion location toward a first target location; store, in the memory, location data of a plurality of points along the first path; receive a selection of at least two points of the plurality of points; edit location data of the selected at least two points; and control steering, during a second navigation, the continuum robot along a second path toward a second target location.

Also provided is a robotic apparatus that includes a continuum robot including a plurality of bending sections, with each section of the plurality of bending sections configured to be bent by at least one wire; a driver configured to drive the at least one wire; a memory; and a controller that is configured to control insertion of the continuum robot into an anatomy; steer, during a first navigation, the continuum robot along a first path toward a target location; store, in the memory, location data of a plurality of points along the first path; control displaying, to a user, the plurality of points along the first path; edit the location data of at least two points selected among the plurality of points; and steer, during a second navigation, the continuum robot along a second path from the target location toward the location of the insertion, with the second path differing from the first path based on the edit of the location data of the selected at least two points.

In addition, a robotic apparatus is provided that includes a continuum robot that includes a continuum robot including a plurality of bending sections, with each section of the plurality of bending sections configured to be bent by at least one wire; a driver configured to drive the at least one wire; a memory; and a controller that is configured to control steering, during a first navigation, the continuum robot along a first path from an insertion of the continuum robot into the anatomy toward a first target location; control storing, in a memory, first location data of a first plurality of points along the first path; control at least partially withdrawing the continuum robot from the anatomy; control steering, during a second navigation, the continuum robot along a second path toward the target location; control storing, in the memory, second location data of a second plurality of points along the second path; determine a center line between the first path and the second path; control at least partially withdrawing the continuum robot from the anatomy; and control steering, during a third navigation, the continuum robot along a third path, with the third path following the determined center line.

Reference numbers:

| | |
|---|---|
| 101 | Display |
| 101-1 | first display |
| 102-2 | second display |
| 100 | display controller |
| 102 | Controller |
| 103 | Actuator |
| 104 | continuum robot/endoscope/ureteroscope |
| 105 | handheld controller |
| 106 | EM tracking sensor |
| 107 | catheter tip position sensor |
| 108 | data storage |
| 109 | insertion/removal detector |
| 110 | ROM |
| 118 | Rail |
| 120 | CPU |
| 130 | RAM |
| 140 | I/O |
| 150 | HDD |
| 304 | tool channel |
| 306 | imaging device |
| 401 | tool insertion slot |

The terms “optical fiber”, “fiber optic”, or simply “fiber” refers to an elongated, flexible, light conducting conduit capable of conducting light from one end to another end due to the effect known as total internal reflection. The terms “light guiding component” or “waveguide” may also refer to, or may have the functionality of, an optical fiber. The term “fiber” may refer to one or more light conducting fibers. An optical fiber has a generally transparent, homogenous core, through which the light is guided, and the core is surrounded by a homogenous cladding. The refraction index of the core is larger than the refraction index of the cladding. Depending on design choice some fibers can have multiple claddings surrounding the core.

The detector interface also provides communication interfaces to input and output devices. The detector may include, for example a photomultiplier tube (PMT), a photodiode, an avalanche photodiode detector (APD), a charge-coupled device (CCD), multi-pixel photon counters (MPPC), or other. Also, the function of detector may be realized by computer executable instructions (e.g., one or more programs) recorded on a storage/RAM.

In referring to the description, specific details are set forth in order to provide a thorough understanding of the examples disclosed. In other instances, well-known methods, procedures, components, and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated herein as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language herein should be construed as indicating any non-claimed element as essential to the practice of the disclosure.

Each patent, patent publication and other publication identified herein and examples described herein are expressly incorporated herein by reference. These publications are provided solely for their disclosure prior to the filing date or applicable priority date of the present application. Nothing in this regard shall be construed as an admission against antedating such publication(s) by virtue of prior invention or other applicable reason. All statements as to the date or representation as to content/disclosure of such publication(s) is based on information available and does not constitute any admission as to accuracy of date of disclosure/publication of such publication(s) or content of such publication(s).

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

What is claimed is:

1. A method for steering a continuum robot, the method comprising:
receiving an identification of a first target location;
inserting the continuum robot;
steering, during a first navigation, the continuum robot along a first path toward the first target location;
storing, in a memory, location data of a plurality of points along the first path;
receiving a selection of at least two points of the plurality of points;
editing location data of the selected at least two points; and
steering, during a second navigation, the continuum robot along a second path toward a second target location,
wherein the second path differs from the first path based on edit of the location data of the selected at least two points.

2. The method of claim 1, wherein the second path is shorter in length than the first path.

3. The method of claim 1, wherein the first target location and the second target location are within an anatomy, and steering during each of the first navigation and the second navigation is performed through the anatomy.

4. The method of claim 1, wherein a tracking sensor is positioned adjacent to a distal end of the continuum robot.

5. The method of claim 4, wherein, during the first navigation, the first location data is obtained from the tracking sensor.

6. The method of claim 1, further comprising:
displaying the plurality of points as a virtual map of the first path.

7. The method of claim 1, further comprising:
interpolating data of points located between the selected at least two points of the first path; and
creating the second path based on the interpolated data.

8. The method of claim 1, wherein, for the plurality of points along the first path, the memory is configured to store at least one of time stamp data, bending angle data, bending plane data, and rail position data with the location data.

9. The method of claim 8, wherein the editing of the first path comprises editing stored time stamp data for each of the at least two selected points.

10. The method of claim 8, wherein the memory is configured to store time stamp data for the plurality of points along the first path with the location data, the method further comprising:
determining, based on the stored time stamp data, locations where the continuum robot is stationary, and
at least one of deleting and/or not storing cumulative location data where the continuum robot is determined to be stationary.

11. The method of claim 10, wherein the memory is configured to store at least one of bending angle information and bending plane information for each point the plurality of points, the method further comprising:
creating the second path by editing the stored at least one of the bending angle information and/or the bending plane information for each point of the selected at least two points of the first path.

12. A method for steering a continuum robot through an anatomy, the method comprising:
inserting the continuum robot into the anatomy;
steering, during a first navigation, the continuum robot along a first path toward a target location;
at least partially withdrawing the continuum robot from the anatomy;
creating a second path using information of the first path;
advancing the continuum robot into the anatomy; and
steering, during a second navigation, the continuum robot along the second path toward the target location,
wherein the second path differs from the first path based on at least one edit of at least two points of the first path.

13. A method for steering a continuum robot, the method comprising:
inserting the continuum robot;
steering, during a first navigation, the continuum robot along a first path from a location of the insertion toward a target location;
storing, in a memory, location data of a plurality of points along the first path;
displaying, to a user, the plurality of points along the first path;
receiving a selection of at least two points among the displayed plurality of points;
editing location data of the selected at least two points; and
steering, during a second navigation, the continuum robot along a second path from the target location toward the location of the insertion,
wherein the second path differs from the first path based on the edit of the location data of the selected at least two points.

14. A method for steering a continuum robot through an anatomy, the method comprising:
inserting the continuum robot into the anatomy;
steering, during a first navigation, the continuum robot along a first path from a location of insertion toward a target location;
storing, in a memory, first location data of a first plurality of points along the first path;
at least partially withdrawing the continuum robot from the anatomy;
steering, during a second navigation, the continuum robot along a second path toward the target location;
storing, in the memory, second location data of a second plurality of points along the second path;
determining a center line between the first path and the second path;
at least partially withdrawing the continuum robot from the anatomy; and
steering, during a third navigation, the continuum robot along a third path,
wherein the third path follows the determined center line.

15. The method of claim 14, wherein the steering during the third navigation is an automated replay function based on an edit of the first location data of at least two points selected among the first plurality of points and an edit of the second location data of at least two points selected among the second plurality of points, and
wherein manual control of the continuum robot is obtained by pausing the automated replay function.

16. A robotic apparatus comprising:
a continuum robot including a plurality of bending sections, with each section of the plurality of bending sections configured to be bent by at least one wire;
a driver configured to drive the at least one wire;
a memory; and
a controller configured to:
control a driving force of the at least one wire;
control steering, during a first navigation, the continuum robot along a first path from an insertion location toward a first target location;
control storing, in the memory, location data of a plurality of points along the first path;
receive a selection of at least two points of the plurality of points;
edit location data of the selected at least two points; and
control steering, during a second navigation, the continuum robot along a second path toward a second target location.

17. The robotic apparatus of claim 16, wherein the second path differs from the first path based on the edit of the location data of the selected at least two points.

18. The robotic apparatus of claim 17, further comprising:
a display; and
an input device,
wherein the controller is further configured to:
control displaying, on the display, a plurality of points forming a path;
receive, from the input device, a selection of at least two points of the plurality of points;
control storing, in the memory, location data with at least one of time stamp data, bending angle data, bending plane data, and rail position data for the first plurality of points; and
editing of the location data of the selected at least two points.

19. A robotic apparatus comprising:
a continuum robot including a plurality of bending sections, with each section of the plurality of bending sections configured to be bent by at least one wire;
a driver configured to drive the at least one wire;
a memory; and
a controller configured to:
control insertion of the continuum robot;
control steering, during a first navigation, the continuum robot along a first path toward a target location;
control storing, in the memory, location data of a plurality of points along the first path;
control displaying, to a user, the plurality of points along the first path;
receive a selection of at least two points among the plurality of points;
edit the location data of at least two selected points; and
control steering, during a second navigation, the continuum robot along a second path from the target location toward the location of the insertion,
wherein the second path differs from the first path based on the edit of the location data of the at least two selected points.

20. A robotic apparatus comprising:
a continuum robot including a plurality of bending sections, with each section of the plurality of bending sections configured to be bent by at least one wire;
a driver configured to drive the at least one wire;
a memory; and
a controller configured to:
control steering, during a first navigation, the continuum robot along a first path from an insertion of the continuum robot toward a target location;
control storing, in a memory, first location data of a first plurality of points along the first path;
control at least partially withdrawing the continuum robot;
control steering, during a second navigation, the continuum robot along a second path toward the target location;
control storing, in the memory, second location data of a second plurality of points along the second path;
determine a center line between the first path and the second path;
control at least partially withdrawing the continuum robot; and
control steering, during a third navigation, the continuum robot along a third path,
wherein the third path follows the determined center line.
